(19) United States
(12) Patent Application Publication
(10) Pub. No.: US 2024/0279610 A1
(43) Pub. Date: Aug. 22, 2024

(54) METHOD FOR IMMORTALISING VESICLE-SECRETING CELLS

(71) Applicant: UNIVERSITÄT DUISBURG-ESSEN, Essen (DE)

(72) Inventors: Bernd GIEBEL, Köln (DE); Helmut HANENBERG, Düsseldorf (DE); Yanis MOULOUD, Essen (DE)

(73) Assignee: UNIVERSITÄT DUISBURG-ESSEN, Essen (DE)

(21) Appl. No.: 18/568,474

(22) PCT Filed: Jun. 9, 2022
(86) PCT No.: PCT/EP2022/065696

§ 371 Date: Dec. 8, 2023

(30) Foreign Application Priority Data
Jun. 9, 2021 (DE) 10 2021 114 823.5

(51) Int. Cl. C12N 5/0775; C12N 9/12; C12N 15/113

(52) U.S. Cl. CPC C12N 5/0662; C12N 9/1276; C12N 15/113; C12Y 207/07049; C12N 2310/14; C12N 2310/531; C12N 2510/04

(57) ABSTRACT

The invention pertains to a method for immortalising cells that are used for the harvesting of extracellular vesicles (EV). EV, derived for example from mesenchymal stromal cells, but also from other cell types, are known to induce beneficial effects during treatment of for example autoimmune disorders. However, the generation of secreting mesenchymal stromal cells is often dependent on donor material or the fact that isolated cells have a limited life span and limited number of passages of cell culture. The present invention provides a method of immortalising such secreting cells, the immortalised cells and medical applications thereof.

### FIELD OF THE INVENTION

The invention pertains to a method for immortalising cells that are used for the harvesting of extracellular vesicles (EVs). EVs, derived for example from mesenchymal stem cells, but also from other cell types, are known to induce beneficial effects during treatment of for example autoimmune disorders. However, the generation of secreting mesenchymal stromal cells is often dependent on donor material or the fact that isolated cells have a limited life span and limited number of passages of cell culture. The present invention provides a method of immortalising such secreting cells, the immortalised cells and medical applications thereof.

### DESCRIPTION

Mesenchymal stem cells (MSCs), also known as mesenchymal stromal cells, are multipotent stem cells that have a limited but robust potential to differentiate into mesenchymal cell types, e.g. adipocytes, chondrocytes and osteocytes. MSC transplantation has been used to treat musculoskeletal injuries, improve cardiac function in cardiovascular disease and ameliorate the severity of graft-versus-host-disease to just mention a few application areas. In recent years, MSC transplantations have demonstrated therapeutic efficacy in treating different diseases but the underlying mechanism has been controversial. Against the initial assumption, MSCs exert their function not by homing to affected organs and tissues to replace lost cell types, but rather improve clinical symptoms by their secretome. This is supported by a variety of observations: MSCs can secrete cytokines and mediate inhibition of T-cell proliferation in the absence of physical intercellular contacts. Furthermore, MSC administration was found to improve induced acute kidney injuries, where the regenerative effect could be attributed to the secretion of insulin-like growth factor-1 (IGF-1). Other examples by which MSCs effect tissue includes the secretion of the anti-inflammatory cytokine TNF-α-induced protein 6 (TNAIP6 or TSG-6). By these mechanisms, MSC-preparations exhibit therapeutic effects on arteriogenesis, stem cell crypt in the intestine, ischemic injury, and hematopoiesis. This paracrine hypothesis could potentially provide for a non-cell based alternative for using MSC in treatment of cardiovascular disease. In support of this paracrine hypothesis, many studies have observed that MSCs secrete cytokines, chemokines and growth factors that could potentially repair injured cardiac tissue mainly through cardiac and vascular tissue growth and regeneration.

Non-cell based therapies as opposed to cell-based therapies are generally easier to manufacture and are safer as they are non-viable and do not elicit immune rejection. It was previously demonstrated that culture medium conditioned by mesenchymal stem cells (MSCs) that were derived from human embryonic stem cells or fetal tissues could protect the heart from myocardial ischemia/reperfusion injury and reduce infarct size. Subsequent studies demonstrated that this cardioprotection was mediated by exosomes or microparticles of about 50-100 nm in diameter and these microparticles carry both protein and RNA. These vesicles could be purified as a population of homogenously sized particles by size exclusion on HPLC and were shown to be therapeutically active.

In a randomized placebo controlled clinical trial performed in Egypt, patients with chronic kidney disease (CKD) stage III and IV have been intravenously treated with two doses of MSC-sEV preparations. Kidney functions were assessed by estimated glomerular filtration rates (eGFR), urinary albumin creatinine ratio, blood urea and serum creatinine levels. Inflammatory immune activities were evaluated by determining blood levels of TNF-α, TGF-β1 and IL-10. Compared to the placebo group, the MSC-sEV treated group exhibited improved CKD symptoms as reflected by creatinine clearance. The improvement was accompanied by significant increases of TGF-β1 and IL-10 concentrations as well as a significant decline of the TNF-α level in the plasma of MSC-sEV treated patients.

The identification of exosomes as the therapeutic agent in the MSC secretion potentially provides for a biologic- rather than cell-based treatment modality. Compared to cellular therapies EV-based therapies provide several hypothetical advantages, e.g. the lack of self-replication potentials of EVs, which in cellular therapies could result in tumour formation. Certain subtypes of EVs, in particular small extracellular vesicles (sEVs) that feature a hydrodynamic diameter of below 200 nm, can be sterilized by filtration. Furthermore, the overall handling and storage is less complicated than for cells. Thus, MSC exosomes (MSC-EVs) provide a promising therapeutic agent for the future. For now, the scalability of the MSC-EV production is limited by the lifespan of MSCs. Since immortalization of MSCs might overcome this issue, we aim to create immortalized MSC lines and test whether their EVs are still therapeutically active

Thus, it is an objection of the invention to provide a method for the generation of a cell line that allows for a continuous harvesting of therapeutically active EVs.

### BRIEF DESCRIPTION OF THE INVENTION

Generally, and by way of brief description, the main aspects of the present invention can be described as follows:

In a first aspect, the invention pertains to a method for immortalising a target cell with a finite life-span, comprising the following steps:
(i) providing the target cell with a finite life-span,
(ii) contacting the target cell with a finite life-span with at least:
   (a) a human Telomerase reverse transcriptase (hTERT) protein, or a genetic expression construct encoding a hTERT protein, and
   (b) one or more inhibitors of the expression, function and/or stability of a cell cycle regulator/tumour suppressor protein;
      Wherein the target cell with a finite life-span is contacted with at least (a) and (b) for a time sufficient to transform the target cell with a finite life-span into an immortalised target cell.

In a second aspect, the invention pertains to an immortalised target cell or immortalised target cell line obtainable with the method of the first aspect.

In a third aspect, the invention pertains to a method for producing a therapeutically active target cell derived extracellular vesicle (EV) preparation, the method comprising:
(i) Providing an immortalised target cell or immortalised target cell line of the second aspect;
(ii) Culturing the immortalised target cell or immortalised target cell line with a culture medium that allow for a secretion of therapeutically modular target cell EVs into the culture medium to obtain an EV enriched medium;
(iii) Harvesting from the EV enriched culture medium the EVs to obtain the therapeutic target cell-derived EV preparation;
(iv) Optionally, testing the therapeutic activity of the therapeutic target cell-derived EV preparation.

In a fourth aspect, the invention pertains to a method for producing a therapeutic target cell derived extracellular vesicle preparation the method comprising:
(i) Providing a target cell with finite life-span and preforming therewith the method of any one of claims 1 to 16 to obtain an immortalised target cell or immortalised target cell line;
(ii) Culturing the immortalised target cell or immortalised target cell line with a culture medium that allow for a secretion of therapeutically modular target cell EVs into the culture medium to obtain an EV enriched medium;
(iii) Harvesting from the EV enriched culture medium the EVs to obtain the therapeutic target cell-derived EV preparation;
(iv) Optionally, testing the immune modulatory activity of the therapeutic target cell-derived EV preparation.

In a fifth aspect, the invention pertains to a vector, or vector set for immortalisation of target cells, the vector or vector set comprising at least the following genetic elements (i) an expression cassette comprising expression control sequences operably linked to at least one hTERT gene, and (ii) an expression cassette comprising expression control sequences operably linked to at least one shRNA-coding sequence, wherein the shRNA targets an mRNA selected from (x) a p21 mRNA, (y) a p16 mRNA, and (z) a p53 mRNA.

In a sixth aspect, the invention pertains to a use of an immortalised target cell or immortalized target cell line of the second aspect, in the manufacturing of a medicament for treatment of an immune mediated, endocrine, orthopaedic, neurodegenerative, cardiovascular and/or respiratory disease or injury.

In a seventh aspect, the invention pertains to a use of an immortalised target cell or immortalised target cell line of the second aspect, in the manufacturing of a medicament for immunomodulatory/immunoregulatory, anti-inflammatory, anti-fibrotic, angiogenetic, and/or regenerative treatment of an injury or a disease.

### DETAILED DESCRIPTION OF THE INVENTION

In the following, the elements of the invention will be described. These elements are listed with specific embodiments; however, it should be understood that they may be combined in any manner and in any number to create additional embodiments. The variously described examples and preferred embodiments should not be construed to limit the present invention to only the explicitly described embodiments. This description should be understood to support and encompass embodiments which combine two or more of the explicitly described embodiments or which combine the one or more of the explicitly described embodiments with any number of the disclosed and/or preferred elements. Furthermore, any permutations and combinations of all described elements in this application should be considered disclosed by the description of the present application unless the context indicates otherwise.

In a first aspect, the invention pertains to a method for immortalising a target cell with a finite life-span, comprising the following steps:
(i) providing the target cell with a finite life-span,
(ii) contacting the target cell with a finite life-span with at least:
   (a) a human Telomerase reverse transcriptase (hTERT) protein, or a genetic expression construct encoding a hTERT protein, and
   (b) one or more inhibitors of the expression, function and/or stability of a cell cycle regulator/tumour suppressor protein;
      Wherein the target cell with a finite life-span is contacted with at least (a) and (b) for a time sufficient to transform the target cell with a finite life-span into an immortalized target cell.

The term “immortalising” a target cell refers to conferring to one or more cells the ability to divide or proliferate to an extent which exceeds its natural, i.e. original proliferation capability, for example within its organism of origin or if isolated and maintained in cell culture. In a preferred embodiment, said cell acquires the ability to divide or proliferate indefinitely. The term “immortalised target cell” also includes post-immortalised cells, i.e. cells which have been immortalised with the method of the invention, but in which proliferation has been terminated or slowed down and/or in which senescence and/or differentiation has been induced. More preferably, said immortalised cells essentially retain the differentiation-specific physiological properties of said cells with a finite life span on which the immortalized cells are based. Essentially retaining the differentiation-specific physiological properties means that the cell or cell line retains at least one property of the cell with a finite life-span which is to be investigated using the immortalised cells. Primarily, this will be related to the primary function(s) of the cell with a finite life-span. Said properties obviously depend on the cell-type and next to the following non-limiting examples, the person skilled in the art can only be referred to methods for examining cell properties known in the art. Immortalized EV secreting cell lines in accordance with the present invention retain at least the property of producing and secreting one or more species of EVs.

The term “cell with a finite life span” refers to any one of the following of (i) non-dividing cells, i.e. cells which do not divide during their life-time, (ii) slowly dividing cells, i.e. cells which in their organism of origin do not have the main or sole purpose of producing other cells by cell division and (iii) cells which senesce, i.e which go through a limited number of cell divisions after isolation from a tissue to stop dividing and/or cells which go into apoptosis i.e primary cells, which directly go into apoptosis after isolation from a tissue or which go through a limited number 20 of cell divisions after isolation from a tissue before going into apoptosis. The term, thus, excludes embryonic stem cells. In a preferred embodiment, it refers to cells derived from the ectoderm, endoderm or mesoderm lineage. Said cells can be growth-arrested cells (e.g. cells which are blocked at various stages of the cell cycle, i.e. G0, G1, S, G2, prophase, prometaphase and metaphase), non-proliferating cells, post- or non-mitotic cells, resting cells, benign cells, senescent cells, in vitro differentiated embryonic stem cells, reprogrammed cells (such as in vitro differentiated induced pluripotent stem cells including their derivatives), terminally differentiated cells, and preferably primary cells. Preferred cells are cells selected from the group consisting of adipocytes, adult and neonatal stem cells, astrocytes, B-cells, cardiomyocytes, chondrocytes, cornea epithelial cells, dendritic cells, endocrine cells, endothelial cells, epithelial cells, fibroblasts, glia cells, granulocytes, hematopoietic cells, hematopoietic stem cells, hepatocytes, keratinocytes, intestinal epithelial cells, liver cells, lung epithelial cells type I, lung epithelial cells type II, lymphocytes, macrophages, mammary epithelial cells, melanocytes, mesangial cells, mesenchymal stem/stromal cells, muscle cells, myoblast, natural killer cells, neuronal cells, neuronal stem cells, neutrophiles, osteoblasts, pancreatic beta cells, pericytes, preadipocytes, progenitor cells, prostate epithelial cells, renal epithelial cells, renal proximal tubule cells, retinal pigment epithelial cells, sertoli cells, skeletal muscle cells, smooth muscle cells, stem cells, stroma cells, T-cells and subsets of said cell types. Said cells are non-mammalian cells (e.g. from fish or bird species) or mammalian cells (e.g. from mice, rats, monkeys, pigs, dogs, cats, cows, sheep, goats), preferably human cells.

Preferably, in one embodiment of the invention, the target cell with finite life-span in step (i) is obtained from biological material including but not limited to bone marrow, adipose tissue, umbilical cord blood, umbilical cord tissue, placenta tissue, periodontal ligament, trabecular bone, synovial membrane, periosteum, muscle and skin tissue.

Most preferably, the target cell with finite life-span is a mesenchymal stem/stromal cell (MSC). Without being bound by theory, it has been suggested that MSC mediated immunosuppression works via paracrine EV-mediated signalling, which include a variety of factors found in secreted vesicles. As used herein, the term “MSC-derived” or “mesenchymal stromal cell-derived” refers to substances that are obtained or derived from a specific source. In the present disclosure, the term “mesenchymal stromal cell” is a multipotent stromal cell that can differentiate into a variety of cell types, including, but not limited to osteoblasts (bone cells), chondrocytes (cartilage cells), myocytes (muscle cells) and adipocytes (fat cells). These cells are also known as “mesenchymal stem cells” due to their multipotency. This biologically important cell population is able to support hematopoiesis, can differentiate along mesenchymal and non-mesenchymal lineages in vitro, is capable of suppressing alloresponses and appear to be non-immunogenic. These cells are known to either secrete certain proteins and/or compounds, which is turn influence the microenvironment around the cells, or when not directly expressing the proteins themselves, mesenchymal stromal cells are known to influence the downstream expression of other proteins, which for example, may be affected by the presence or absence of a mesenchymal stromal cell.

In one preferred embodiment of the invention, the method of the first aspect is a non-therapeutic, non-diagnostic and non-surgical method. More preferably the method of the first aspect is conducted entirely ex vivo or in vitro.

It is a surprisingly result of the present invention that a preferred combination of hTERT expression and inhibition of a cell cycle regulator/tumour suppressor protein results in the immortalisation of the EV secreting target cell without affecting its EV-secreting ability.

The immortalisation of the invention employs the recombinant expression of the human telomerase (hTERT) It acts by maintaining the ends of the telomeres, which are stretches of repetitive DNA at the very end of the linear chromosomes. These stretches cannot be replicated by DNA polymerases during replication and therefore the telomeres progressively shorten with every replication round. This “end-replication problem” can be overcome by the recombinant expression of hTERT which eventually can lead to immortalisation. Cell types expanded and used for tissue engineering include e.g. bovine adrenocortical cells (reference), human dermal endothelial cells (Yang J et al., Nat Biotechnol. March 2001; 19(3):219-224) and human MSCs (Simonsen J L et al., Nat Biotechnol. June 2002; 20(6):592-596). However, prolonged constitutive expression of telomerase induces changes in gene expression that lead to a premalignant phenotype (Milyaysky M, et al., Cancer Res. Nov. 1, 2003; 63(21):7147-7157). In addition, the use of hTert is restricted to certain human cell types as others need the concerted action of several genes for efficient immortalisation (Kiyono T et al., Nature. Nov. 5, 1998; 396(6706):84-88). In the context of the present invention human Telomerase reverse transcriptase (hTERT) protein is a protein shown in the UniProt database under the accession no. O14746 (https://www.uniprot.org/uniprot/O14746—database version of February 2021). Four isoforms of human TERT are known which are included herein as SEQ ID NO: 1 to 4 respectively.

The term cell cycle regulator/tumour suppressor protein is a physiological, optionally endogenous, substance actively involved in the cell cycle control of a eukaryotic cell and in context of the present invention preferably selected from p21, p16 and p53. Preferred is in context of the invention, that step (ii) of the method of the first aspect comprises contacting the target cell with finite life-span with at least one of (a) and with at least one or more inhibitor of the expression, function and/or stability in (b).

The term “p21” in context of the invention refers to a protein known as Cyclin-dependent kinase inhibitor 1, expressed and encoded by the gene CDKN1A. P21 may be involved in p53/TP53 mediated inhibition of cellular proliferation in response to DNA damage. It binds to and inhibits cyclin-dependent kinase activity, preventing phosphorylation of critical cyclin-dependent kinase substrates and blocking cell cycle progression. Functions in the nuclear localization and assembly of cyclin D-CDK4 complex and promotes its kinase activity towards RB1. At higher stoichiometric ratios, inhibits the kinase activity of the cyclin D-CDK4 complex. P21 inhibits DNA synthesis by DNA polymerase delta by competing with POLD3 for PCNA binding (PubMed: 11595739). Plays an important role in controlling cell cycle progression and DNA damage-induced G2 arrest. The p21 protein can be derived from the UniProt database under the accession no. P38936 (https://www.uniprot.org/uniprot/P38936—database version of February 2021).

The term “p16” in context of the invention refers to p16 protein (also known as p16INK4a, cyclin-dependent kinase inhibitor 2A, CDKN2A, multiple tumor suppressor 1 and numerous other synonyms), which is a protein that slows cell division by slowing the progression of the cell cycle from the G1 phase to the S phase, thereby acting as a tumor suppressor. It is encoded by the CDKN2A gene. The p16 protein can be derived from the UniProt database under the accession no. P42771 (https://www.uniprot.org/uniprot/P42771—database version of February 2021).

The term “p53” in context of the invention refers to a protein that acts as a tumor suppressor in many tumor types; induces growth arrest or apoptosis depending on the physiological circumstances and cell type. It is involved in cell cycle regulation as a trans-activator that acts to negatively regulate cell division by controlling a set of genes required for this process. The p53 protein can be derived from the UniProt database under the accession no. P04637 (https://www.uniprot.org/uniprot/P04637—database version of February 2021).

In context of the present invention variants of the aforementioned human proteins are included in some embodiments. Such variants are preferably proteins having equal to or more than 80% sequence identity to the amino acid sequence of the respective human counterpart. Also included in some embodiments are orthologues, paralogues or homologues of the respective proteins.

In one particular set of embodiments, the inhibitors used in context of the present invention are nucleic acid inhibitors, such as preferably antisense constructs.

The terms “nucleic acid”, “polynucleotide” and “oligonucleotide” are used interchangeably throughout and include DNA molecules (e.g., cDNA or genomic DNA), RNA molecules (e.g., mRNA), analogues of the DNA or RNA generated using nucleotide analogues (e.g., peptide nucleic acids and non-naturally occurring nucleotide analogues), and hybrids thereof. The nucleic acid molecule can be single-stranded or double-stranded.

In particular embodiments, the inhibitor may be an inhibitory nucleic acid molecule, such as antisense nucleotide molecule including a siRNA or shRNA molecule, for example as described in detail herein below. In more particular of such embodiments, the inhibitory nucleic acid (such as siRNA or shRNA) can bind to, such as specifically bind to, a nucleic acid (such as mRNA) that encodes or regulates the expression, amount, function, activity or stability of: (i) p21, p16 and/or P53.

An inhibitor of p21, p16 and/or p53 that is a nucleic acid can be, for example, an anti-sense nucleotide molecule, an RNA, DNA or PNA molecule, or an aptamer molecule. An anti-sense nucleotide molecule can, by virtue of it comprising an anti-sense nucleotide sequence, bind to a target nucleic acid molecule (eg based on sequence complementarity) within a cell and modulate the level of expression (transcription and/or translation) of p21, p16 and/or p53, or it may modulate expression of another gene that controls the expression, function and/or stability of p21, p16 and/or p53. Similarly, an RNA molecule, such as a catalytic ribozyme, can bind to and alter the expression of the p21, p16 and/or p53 gene, or it can bind to and alter the expression of other genes that control the expression, function and/or stability of p21, p16 and/or p53, such as a transcription factor for or repressor protein of p21, p16 and/or p53. An aptamer is a nucleic acid molecule that has a sequence that confers it an ability to form a three-dimensional structure capable of binding to a molecular target.

An inhibitor of p21, p16 and/or p53 that is a nucleic acid can be, for example, a double-stranded RNA molecule for use in RNA interference. RNA interference (RNAi) is a process of sequence-specific gene silencing by post-transcriptional RNA degradation or silencing (prevention of translation). RNAi is initiated by use of double-stranded RNA (dsRNA) that is homologous in sequence to the target gene to be silenced. A suitable double-stranded RNA (dsRNA) for RNAi contains sense and antisense strands of about 21 contiguous nucleotides corresponding to the gene to be targeted that form 19 RNA base pairs, leaving overhangs of two nucleotides at each 3′ end (Elbashir et al., Nature 411:494-498 (2001); Bass, Nature 411:428-429 (2001); Zamore, Nat. Struct. Biol. 8:746-750 (2001)). dsRNAs of about 25-30 nucleotides have also been used successfully for RNAi (Karabinos et al., Proc. Natl. Acad. Sci. USA 98:7863-7868 (2001). dsRNA can be synthesised in vitro and introduced into a cell by methods known in the art.

A particularly preferred example of an antisense molecule of the invention is a small interfering RNA (siRNA) or endoribonuclease-prepared siRNA (esiRNA). An esiRNA is a mixture of siRNA oligos resulting from cleavage of a long double-stranded RNA (dsRNA) with an endoribonuclease such as Escherichia coli RNase III or dicer. esiRNAs are an alternative concept to the usage of chemically synthesised siRNA for RNA Interference (RNAi). An esiRNAs is the enzymatic digestion of a long double stranded RNA in vitro.

As described above, a modulator of the invention that is an RNAi molecule (such as an siRNA) may bind to and directly inhibit or antagonise the expression of mRNA of p21, p16 and/or p53. However, a modulator of the invention that is an RNAi molecule (such as an siRNA) may bind to and inhibit or antagonise the expression of mRNA of another gene that itself controls the expression (or function or stability) of p21, p16 and/or p53.

The sequence identity of the antisense molecule according to the invention in order to target a p21, p16 and/or p53 mRNA (or to target mRNA of a gene controlling expression, function and/or stability of p21, p16 and/or p53), is with increasing preference at least 75%, at least 80%, at least 85%, at least 90%, at least 95%, at least 98%, at least 99% and 100% identity to a region of a sequence encoding the p21, p16 and/or p53 protein, as disclosed. Preferably, the region of sequence identity between the target gene and the modulating antisense molecule is the region of the target gene corresponding to the location and length of the modulating antisense molecule. For example, such a sequence identity over a region of about 19 to 21 bp of length corresponding to the modulating siRNA or shRNA molecule). Means and methods for determining sequence identity are known in the art. Preferably, the BLAST (Basic Local Alignment Search Tool) program is used for determining the sequence identity with regard to one or more p21, p16 and/or p53 RNAs as known in the art. On the other hand, preferred antisense molecules such as siRNAs and shRNAs of the present invention are preferably chemically synthesised using appropriately protected ribonucleoside phosphoramidites and a conventional RNA synthesiser. Suppliers of RNA synthesis reagents include Proligo (Hamburg, Germany), Dharmacon Research (Lafayette, CO, USA), Pierce Chemical (part of Perbio Science, Rockford, IL, USA), Glen Research (Sterling, VA, USA), ChemGenes (Ashland, MA, USA), and Cruachem (Glasgow, UK). Preferably, the antisense construct is selected from an RNA interference inducing nucleic acid, such as an siRNA or shRNA, and preferably is an shRNA.

As mentioned, the inhibition of p21, p16 and/or p53 in the target vesicle secreting cell is preferably done using an shRNA molecule. As used herein, an “shRNA molecule” includes a conventional stem-loop shRNA, which forms a precursor miRNA (pre-miRNA). “shRNAs” also includes micro-RNA embedded shRNAs (miRNA-based shRNAs), wherein the guide strand and the passenger strand of the miRNA duplex are incorporated into an existing (or natural) miRNA or into a modified or synthetic (designed) miRNA. When transcribed, an shRNA forms a primary miRNA (pri-miRNA) or a structure very similar to a natural pri-miRNA. The pri-miRNA is subsequently processed by Drosha and its cofactors into pre-miRNA. Therefore, the term “shRNA” includes pri-miRNA (shRNA-mir) molecules and pre-miRNA molecules.

A “stem-loop structure” refers to a nucleic acid having a secondary structure that includes a region of nucleotides which are known or predicted to form a double strand or duplex (stem portion) that is linked on one side by a region of predominantly single-stranded nucleotides (loop portion). The terms “hairpin” and “fold-bac” structures are also used herein to refer to stem-loop structures. Such structures are well known in the art and the term is used consistently with its known meaning in the art. As is known in the art, the secondary structure does not require exact base-pairing. Thus, the stem can include one or more base mismatches or bulges. Alternatively, the base-pairing can be exact, i.e., not include any mismatches.

In some instances, the precursor miRNA molecule can include more than one stem-loop structure. The multiple stem-loop structures can be linked to one another through a linker, such as, for example, a nucleic acid linker, a miRNA flanking sequence, other molecule, or some combination thereof.

MicroRNAs are endogenously encoded RNA molecules that are about 22-nucleotides long and generally expressed in a highly tissue- or developmental-stage-specific fashion and that post-transcriptionally regulate target genes. More than 200 distinct miRNAs have been identified in plants and animals. These small regulatory RNAs are believed to serve important biological functions by two prevailing modes of action: (1) by repressing the translation of target mRNAs, and (2) through RNA interference (RNAi), that is, cleavage and degradation of mRNAs. In the latter case, miRNAs function analogously to small interfering RNAs (siRNAs). The highly tissue-specific or developmentally regulated expression of miRNAs is likely key to their predicted roles in eukaryotic development and differentiation. Analysis of the endogenous role of miRNAs is facilitated by techniques that allow the regulated over-expression or inappropriate expression of authentic miRNAs in vivo. The ability to regulate the expression of siRNAs will greatly increase their utility both in cultured cells and in vivo. Thus, one can design and express artificial miRNAs based on the features of existing miRNA genes. Short hairpin RNAs can be designed to mimic endogenous miRNAs. Many miRNA intermediates can be used as models for shRNA or shRNAmir, including without limitation a miRNA comprising a backbone design of miR-15a, -16, -19b, -20, -23a, -27b, -29a, -30b, -30c, -104, -132s, -181, -191, -223 (see U.S. Publication No. 2005/0075492). The miR-30 natural configuration has proven especially beneficial in producing mature synthetic miRNAs. miR30-based shRNAs and shRNAmirs have complex folds, and, compared with simpler stem/loop style shRNAs, are more potent at inhibiting gene expression in transient assays.

In context of the invention the inhibition of p21, p16 and/or p53 in the target EV secreting cell is preferably realized by introducing a genetic expression construct into the target vesicle secreting cell that is suitable for a constitutive, or preferably inducible, expression of the RNA interference molecule, such as the shRNA molecule, in the target cell. An “RNAi-expressing construct” or “RNAi construct” is a generic term that includes nucleic acid preparations designed to achieve an RNA interference effect in a target cell. An RNAi-expressing construct comprises an RNAi molecule that can be cleaved in vivo to form an siRNA. For example, an RNAi construct is an expression vector capable of giving rise to an siRNA or an shRNA in vivo. Exemplary methods of making and delivering long or short RNAi constructs can be found, for example, in U.S. Patent Application Nos. 2002/008635 and 2004/0018999. In certain embodiments of the invention, such as those directed to therapeutic applications, it may be desirable to use siRNAs, including modified siRNAs, based on the sequences of shR As described herein. For example, it may be desirable to use an siRNA that binds to the same target sequence as the shRNA sequences disclosed herein. One of skill in the art can readily design such siRNAs, for example basing the siRNA sequence on any or all parts of the shRNA sequence that is complementary to or binds to the target sequence.

In certain embodiments, useful interfering RNAs can be designed with a number of software programs, e.g., the OligoEngine siRNA design tool. Algorithms for in silico prediction, or algorithms based on an empirically trained neural network, such as BIOPREDsi, can be used. Birmingham et al. (2007, Nat. Protocols 2: 2068-2078) provide a comprehensive overview of prediction algorithms.

shRNA can be expressed from suitable expression vectors to provide sustained silencing and high yield delivery into almost any cell type. Where the objective is to identify new therapeutic targets, the shRNAs may be programmed for inducible expression. Where the objective is therapeutic, the shRNAs may be programmed for constitutive and/or cell type specific expression.

Essentially any method for introducing an shRNA expression construct into cells can be employed. Physical methods of introducing nucleic acids include injection of a solution containing the construct, bombardment by particles covered by the construct, soaking a cell, tissue sample or organism in a solution of the nucleic acid, or electroporation of cell membranes in the presence of the construct. A viral construct packaged into a viral particle can be used to accomplish both efficient introduction of an expression construct into the cell and transcription of the encoded shRNA. Other methods known in the art for introducing nucleic acids to cells can be used, such as lipid-mediated carrier transport, chemical mediated transport, such as calcium phosphate, and the like. Thus, the shRNA-encoding nucleic acid construct can be introduced along with components that perform one or more of the following activities: enhance RNA uptake by the cell, promote annealing of the duplex strands, stabilize the annealed strands, or otherwise increase inhibition of the target gene. Cells transduced with the shRNA expression constructs according to the methods described herein may be introduced into a mammal (or used to generate a mammal) and propagated in vivo without significant loss of the RNA interference effects in the cells or their progeny.

Viral or non-viral vectors may be used for expressing shRNAs. In one embodiment, the vector is a viral vector. Viral vector-based RNAi delivery not only allows for stable single-copy genomic integrations but also avoids the non-sequence specific response via cell-surface toll-like receptor 3 (TLR3), which has raised many concerns for the specificity of siRNA mediated effects. Viral vectors may be derived from a retrovirus (including a lentivirus, such as HIV-1 and HIV-2), adeno-associated virus (AAV), adenovirus, herpesvirus, vaccinia virus, poliovirus, poxvirus, Sindbis virus, other RNA viruses, including alphaviruses, astroviruses, coronaviruses, paramyxoviruses, orthomyxoviruses, picornaviruses, and togaviruses; other DNA viruses, including papovaviruses, parvoviruses, and the like.

Lentiviral systems permit the delivery and expression of shRNA constructs to both dividing and non-dividing cell populations in vitro and in vivo. Exemplary lentiviral vectors include those based on HIV, FIV and EIAV. See, e.g., Lois, C, et al., Science, 2002, 295(5556): p. 868-72. Most viral systems contain cis-acting elements necessary for packaging, while trans-acting factors are supplied by a separate plasmid that is co-transfected with the vector into a packaging cell line. In certain embodiments, a highly transfectable 293 cell line may be used for packaging vectors, and viruses may be pseudotyped with a Foamy Virus envelope glycoprotein for enhanced stability and to provide broad host range for infection. In certain aspects, the invention provides novel vectors adapted for use with shRNA expression cassettes. For example, a Gateway recipient sequence may be inserted downstream of the packaging signal to facilitate movement of the shRNA construct to and from different vector backbones by simple recombination. As another example, recombination signals may be inserted to facilitate in vivo transfer of shRNAs from, e.g., a genome-wide shRNA library. Other retroviral expression vector systems for expression of shRNAs include, but are not limited to, Moloney murine leukemia virus, spleen necrosis virus, Rous sarcoma virus, Harvey murine sarcoma virus, avian leukosis virus, gibbon ape leukemia virus, human immunodeficiency virus, myeloproliferative sarcoma virus, and mammary tumor virus. A retroviral plasmid vector can be employed to transduce packaging cell lines to form producer cell lines. Examples of packaging cells which can be transfected include, but are not limited to, the PE501, PA317, R-2, R-AM, PA12, T19-14X, VT-19-17-H2, RCRE, RCRIP, GP+E-86, GP+envAml2, and DAN cell lines. The vector may transduce the packaging cells through any means known in the art. A producer cell line generates infectious retroviral vector particles which include polynucleotide encoding a polypeptide of the present invention. Such retroviral vector particles then may be employed, to transduce eukaryotic cells in vitro, in vivo or ex vivo so as to express an shRNA in accordance with the present application.

In certain embodiments, cells can be engineered using an adeno-associated virus (AAV). AAVs are naturally occurring defective viruses that require helper viruses to produce infectious particles. They are also one of the few viruses that can integrate its DNA into nondividing cells. Vectors containing as little as 300 base pairs of AAV can be packaged and can integrate, but space for exogenous DNA is limited to about 4.5 kb. Methods for producing and using such AAVs are known in the art. See e.g., U.S. Pat. Nos. 5,139,941, 5,173,414, 5,354,678, 5,436,146, 5,474,935, 5,478,745, and 5,589,377. For example, an AAV vector can include all the sequences necessary for DNA replication, encapsidation, and host-cell integration. The recombinant AAV vector can be transfected into packaging cells which are infected with a helper virus, using any standard technique, including lipofection, electroporation, calcium phosphate precipitation, etc. Appropriate helper viruses include adenoviruses, cytomegaloviruses, vaccinia viruses, or herpes viruses. Once the packaging cells are transfected and infected, they will produce infectious AAV viral particles which contain the polynucleotide construct. These viral particles are then used to transduce eukaryotic cells. Exemplary vectors for expressing shRNAs are described in U.S. Patent Publication No. 201 10015093.

Additional viral vectors include recombinant parainfluenza virus (PIV) vectors as disclosed in e.g., U.S. Patent Publication No. 2003/0232326 and recombinant metapneumovirus virus (MPV) vectors as disclosed in e.g., U.S. Patent Publication No. 2004/0005544.

A non-viral vector is simply a “naked” expression vector that is not packaged with virally derived components (e.g., capsids and/or envelopes). Non-viral expression vectors may encapsulate the shRNA expression constructs in liposomes, microparticles, microcapsules, virus-like particles, or erythrocyte ghosts. Such compositions can be further linked by chemical conjugation to targeting domains to facilitate targeted delivery and/or entry of nucleic acids into desired cells of interest. In addition, plasmid vectors may be incubated with synthetic gene transfer molecules such as polymeric DNA-binding cations like polylysine, protamine, and albumin, and linked to cell targeting ligands such as asialoorosomucoid, insulin, galactose, lactose or transferrin.

Alternatively, transfection of naked DNA may be employed. Uptake efficiency of naked DNA may be improved by compaction or by using biodegradable latex beads. Such delivery may be improved further by treating the beads to increase hydrophobicity and thereby facilitate disruption of the endosome and release of the DNA into the cytoplasm.

In certain preferred embodiments, the coding sequence of the RNAi molecule is controlled by a tetracycline-responsive promoter. See U.S. Pat. Publ. No. 2008/0226553. The TET system is a preferred system for the present invention. Tetracycline (Tet)-responsive promoters can be used for in vitro and in vivo studies. Tet-On is a variation of the Tet-Off system, which features a modified Tet repressor that has reversed DNA binding properties when compared to the wild-type Tet-repressor (tetR) encoded in the TnlO Tet-resistance operon of E. coli. The reverse tetracycline-controlled transactivator (rtTA) is made from a Tet-repressor fused to the activating domain of virion protein 16 (VP 16) of herpes simplex virus (HSV). In contrast to the Tet-Off system, the Tet-On system is optimized for induction by the Tet-analogue doxycycline (Dox) only. Expression of rtTA can be driven by a constitutive promoter of choice. When rtTA is expressed, the presence of Dox leads to a conformational change and binding of rtTA-Dox to the Tet operator sequence (tetO) of the Tet-resistance operon. The rtTA3 is an improved variant of the reverse tet-trans activator, showing a more sigmoidal induction curve, which is a result of less background activity Off-Dox (tet-On system) and full induction of transgene expression at lower doxycycline (Dox) concentrations (Urlinger et al., (2000), Proc. Natl. Acad. Sci. U.S.A. 97, 7963-7968). Seven serial tetO sequences were fused to a minimal cytomegalovirus (CMV) promoter and termed the Tet-responsive element (TRE). The binding of rtTA-Dox, therefore, induces the expression of a gene of interest from the minimal CMV promoter. Thus, by placing an shRNA under the control of the TRE, the expression of the RNAi molecule is inducible by the addition of Dox.

To facilitate RNA-mediated inhibition in a cell line or whole organism, or monitor the target gene knockdown and its effect on the progression of inflammatory bowel disease, cancer, or any other disease condition, cells harboring the RNAi-expressing construct can additionally comprise a marker or reporter construct, such as a fluorescent construct. In particular, gene expression can be conveniently assayed using a reporter or drug resistance gene whose protein product is easily assayed. Further, when conditional expression of the RNAi construct is coupled to the expression/activity of certain markers, such as a visible GFP marker, the system allows for monitoring of RNAi production in live animals without having to sacrifice the animals, thus live disease progression and the time course of host response to therapy can be monitored in real time. In addition, an shRNA marker construct can be used as a control for inducible expression and to verify that the backbone in the target shRNA of interest is not toxic.

si- and shRNAs targeting genes of interest can be chosen from various sources. In one embodiment, the RNAi sequences are selected from existing libraries. For example, Silva et al. (2005, Nat. Genet. 37: 1281-1288), have described extensive libraries of pri-miR-30-based retroviral expression vectors that can be used to down-regulate almost all known human (at least 28,000) and mouse (at least 25,000) genes (see RNAi CODEX, a single database that curates publicly available RNAi resources, and provides the most complete access to this growing resource, allowing investigators to see not only released clones but also those that are soon to be released, available at the Cold Spring Harbor Laboratories website). Pools of shRNAs useful to practice methods of the invention can be from the “the Cancer 1000” library, which was constructed by Steve Elledge and Greg Hannon. The “Cancer 1000” shRNA library includes a mixture of well characterized oncogenes and tumor suppressor genes in addition to many poorly-characterized genes somehow related to cancer, across many ontological groups, as compiled by literature mining.

In a preferred embodiment of the invention, the herein disclosed method of the first aspect in step (ii) the target cell with finite life-span is contacted with the genetic expression construct, such as a lentiviral expression construct, encoding the hTERT protein and at least one shRNA expression construct encoding at least one shRNA comprising a sequence hybridizing to an mRNA selected from (x) a p21 mRNA, (y) a p16 mRNA, and (z) a p53 mRNA, preferably, thereby inhibiting the protein expression of p21, p16 and/or p53 in the target cell with finite life-span.

The components for immortalising the target cells according to the invention in some embodiments may be provided as individual genetic constructs, or alternatively may be provided in one genetic construct having multiple expression cassettes which are suitable for the expression of the hTERT and the one or more inhibitors, such as shRNA preferably, of p21 mRNA, a p16 mRNA, and a p53 mRNA.

The genetic expression constructs used in context of the invention, preferably such encoding the hTERT protein and the at least one inhibitor of the expression, function and/or stability of a cell cycle regulator/tumour suppressor protein are inducible genetic expression constructs. Preferably the genetic expression constructs are controlled by a tetracycline-controlled operator system as described herein above for the RNAi constructs. In such embodiments wherein an inducible expression system, in particular of the RNAi constructs, is used, the method may further comprise a step of stopping the expression of the one or more RNAi constructs, therefore reducing or switching off the expression of the inhibitor expression, in order to proceed with the so immortalised cells to a harvesting phase. By reducing the RNAi expression for the harvesting phase of secreted vesicles, it can be avoided that possible tumour inducing RNA has to be separated from the vesicles composition.

Further additional steps of the method may include subsequent to step (ii) an optional step (iii) of terminating or slowing down proliferation, inducing senescence and/or inducing differentiation in the immortalised target cell.

Preferably, the one or more expression construct in accordance with the invention is stably or transiently introduced into the target cell with finite life-span. Stable introduction may be achieved by using a vector that mediates the introduction of the genetic construct into the genome of the target vesicle secreting cell. Transient introduction is achieved using vectors which remain extra-chromosomal and dilute in the process of cell division. However, in context of the present invention the stable introduction is a preferred embodiment.

It is preferred, that the genetic expression construct and the at least one inhibitor of the expression, function and/or stability of a cell cycle regulator/tumour suppressor protein are expressed simultaneously in the target vesicle secreting cell.

In the event that the EVs obtained from the cells of the invention are used for therapy of a subject, said target cell with finite life-span is derived from an individual or a group of individuals which are intended to be treated with an EV preparation obtained by culturing the immortalised target cell. Hence, the methods of the invention may be used in the context of a cell autologous situation where a patient may receive vesicles obtained from immortalised self-autologous cells which were produced according to the invention.

In a second aspect, the invention pertains to an immortalised target cell or immortalised target cell line obtainable with the method of the first aspect.

In a third aspect, the invention pertains to a method for producing a therapeutically active target cell derived EV preparation, the method comprising:
(i) Providing an immortalised target cell or immortalised target cell line of the second aspect;
(ii) Culturing the immortalised target cell or immortalised target cell line with a culture medium that allow for a secretion of therapeutically modular target cell EVs into the culture medium to obtain an EV enriched medium;
(iii) Harvesting from the EV enriched culture medium the EVs to obtain the therapeutic target cell-derived EV preparation;
(iv) Optionally, testing the therapeutic activity of the therapeutic target cell-derived EV preparation.

In a fourth aspect, the invention pertains to a method for producing a therapeutic target cell derived extracellular vesicle (EV) preparation the method comprising:
(i) Providing a target cell with finite life-span and preforming therewith the method of any one of claims 1 to 16 to obtain an immortalised target cell or immortalised target cell line;
(ii) Culturing the immortalised target cell or immortalised target cell line with a culture medium that allow for a secretion of therapeutically modular target cell EVs into the culture medium to obtain an EV enriched medium;
(iii) Harvesting from the EV enriched culture medium the EVs to obtain the therapeutic target cell-derived EV preparation;
(iv) Optionally, testing the immune modulatory activity of the therapeutic target cell-derived EV preparation.

In a fifth aspect, the invention pertains to a vector, or vector set for immortalisation of target cells, the vector or vector set comprising at least the following genetic elements (i) an expression cassette comprising expression control sequences operably linked to at least one hTERT gene, and (ii) an expression cassette comprising expression control sequences operably linked to at least one shRNA-coding sequence, wherein the shRNA targets an mRNA selected from (x) a p21 mRNA, (y) a p16 mRNA, and (z) a p53 mRNA.

In a sixth aspect, the invention pertains to a use of an immortalised target cell or immortalized target cell line of the second aspect, in the manufacturing of a medicament for treatment of an immune mediated, endocrine, orthopaedic, neurodegenerative, cardiovascular and/or respiratory disease or injury.

In a seventh aspect, the invention pertains to a use of an immortalised target cell or immortalised target cell line of the second aspect, in the manufacturing of a medicament for immunomodulatory/immunoregulatory, anti-inflammatory, anti-fibrotic, angiogenetic, and/or regenerative treatment of an injury or a disease.

The terms “of the [present] invention”, “in accordance with the invention”, “according to the invention” and the like, as used herein are intended to refer to all aspects and embodiments of the invention described and/or claimed herein.

As used herein, the term “comprising” is to be construed as encompassing both “including” and “consisting of”, both meanings being specifically intended, and hence individually disclosed embodiments in accordance with the present invention. Where used herein, “and/or” is to be taken as specific disclosure of each of the two specified features or components with or without the other. For example, “A and/or B” is to be taken as specific disclosure of each of (i) A, (ii) B and (iii) A and B, just as if each is set out individually herein. In the context of the present invention, the terms “about” and “approximately” denote an interval of accuracy that the person skilled in the art will understand to still ensure the technical effect of the feature in question. The term typically indicates deviation from the indicated numerical value by ±20%, ±15%, ±10%, and for example ±5%. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect. Where an indefinite or definite article is used when referring to a singular noun, e.g. “a”, “an” or “the”, this includes a plural of that noun unless something else is specifically stated.

It is to be understood that application of the teachings of the present invention to a specific problem or environment, and the inclusion of variations of the present invention or additional features thereto (such as further aspects and embodiments), will be within the capabilities of one having ordinary skill in the art in light of the teachings contained herein.

Unless context dictates otherwise, the descriptions and definitions of the features set out above are not limited to any particular aspect or embodiment of the invention and apply equally to all aspects and embodiments which are described.

All references, patents, and publications cited herein are hereby incorporated by reference in their entirety.

### BRIEF DESCRIPTION OF THE FIGURES AND SEQUENCES

The figures show:

FIG. 1: The immortalized MSC cell line can grow and proliferate over 15 passages whereas primary ones reach a maximum of 6 passages.

FIG. 2: (A) Non-immortalized primary MSC and Immortalized MSC cell line have the same morphology; (B) The immortalized MSC cell line shows the same molecular cell surface phenotype as (C) the original primary MSC.

FIG. 3: The immunomodulatory effect of MSC-EV preparations from immortalized MSCs. The plots show each show the fraction of activated immune cells from samples exposed to a given amount of MSC-EVs as determined by the mixed lymphocyte reaction assay of WO 2014/013029. The effect of the MSC-EV preparations is shown in (A) for CD4+ (CD54/CD25ghigh), in (B) for CD8+(CD54/CD25high) and in (C) for different monocyte subsets. The immunomodulatory activity of immortalized MSC which as primary MSCs regularly produced immunomodulatory active EVs, can only be seen when they are used in high doses.

FIG. 4: Shown is the immunomodulatory activity of MSC-EVs prepared from cell culture supernatants of different clonal cultures from immortalized MSCs. All clones secret MSC-EVs with similar immunomodulatory potential that is comparable to the immunomodulatory potential of the EVs of the parental immortalized MSC cells. (A and B) shows the effect of MSC EVs on CD4+ T cells, (C and D) shows the effect of MSC-EVs on CD8+ T cells, and (E and F) shows the effect of MSC EVs on various monocyte subtypes.

The sequences show:

```
hTERT isoform 1
SEQ ID NO. 1
                10 20 30 40 50
MPRAPRCRAV RSLLRSHYRE VLPLATFVRR LGPQGWRLVQ RGDPAAFRAL
                60 70 80 90 100
VAQCLVCVPW DARPPPAAPS FRQVSCLKEL VARVLQRLCE RGAKNVLAFG
              110 120 130 140 150
FALLDGARGG PPEAFTTSVR SYLPNTVTDA LRGSGAWGLL LRRVGDDVLV
              160 170 180 190 200
HLLARCALFV LVAPSCAYQV CGPPLYQLGA ATQARPPPHA SGPRRRLGCE
              210 220 230 240 250
RAWNHSVREA GVPLGLPAPG ARRRGGSASR SLPLPKRPRR GAAPEPERTP
              260 270 280 290 300
VGQGSWAHPG RTRGPSDRGF CVVSPARPAE EATSLEGALS GTRHSHPSVG
              310 320 330 340 350
RQHHAGPPST SRPPRPWDTP CPPVYAETKH FLYSSGDKEQ LRPSFLLSSL
              360 370 380 390 400
RPSLTGARRL VETIFLGSRP WMPGTPRRLP RLPQRYWQMR PLFLELLGNH
              410 420 430 440 450
AQCPYGVLLK THCPLRAAVT PAAGVCAREK PQGSVAAPEE EDTDPRRLVQ
              460 470 480 490 500
LLRQHSSPWQ VYGFVRACLR RLVPPGLWGS RHNERRFLRN TKKFISLGKH
              510 520 530 540 550
AKLSLQELTW KMSVRDCAWL RRSPGVGCVP AAEHRLREEI LAKFLHWLMS
              560 570 580 590 600
VYVVELLRSF FYVTETTFQK NRLFFYRKSV WSKLQSIGIR QHLKRVQLRE
              610 620 630 640 650
LSEAEVRQHR EARPALLTSR LRFIPKPDGL RPIVNMDYVV GARTFRREKR
              660 670 680 690 700
AERLTSRVKA LFSVLNYERA RRPGLLGASV LGLDDIHRAW RTFVLRVRAQ
              710 720 730 740 750
DPPPELYFVK VDVTGAYDTI PQDRLTEVIA SIIKPQNTYC VRRYAVVQKA
              760 770 780 790 800
AHGHVRKAFK SHVSTLTDLQ PYMRQFVAHL QETSPLRDAV VIEQSSSLNE
              810 820 830 840 850
ASSGLFDVFL REMCHHAVRI RGKSYVQCQG IPQGSILSTL LCSLCYGDME
              860 870 880 890 900
NKLFAGIRRD GLLLRLVDDF LLVTPHLTHA KTFLRTLVRG VPEYGCVVNL
              910 920 930 940 950
RKTVVNFPVE DEALGGTAFV QMPAHGLFPW CGLLLDTRTL EVQSDYSSYA
              960 970 980 990 1000
RTSIRASLTF NRGFKAGRNM RRKLFGVLRL KCHSLFLDLQ VNSLQTVCTN
              1010 1020 1030 1040 1050
IYKILLLQAY RFHACVLQLP FHQQVWKNPT FFLRVISDTA SLCYSILKAK
            1060 1070 1080 1090 1100
NAGMSLGAKG AAGPLPSEAV QWLCHQAFLL KLTRHRVTYV PLLGSLRTAQ
            1110 1120 1130
TQLSRKLPGT TLTALEAAAN PALPSDFKTI LD
hTERT isoform 2
SEQ ID NO. 2
                10 20 30 40 50
MPRAPRCRAV RSLLRSHYRE VLPLATFVRR LGPQGWRLVQ RGDPAAFRAL
                60 70 80 90 100
VAQCLVCVPW DARPPPAAPS FRQVSCLKEL VARVLQRLCE RGAKNVLAFG
              110 120 130 140 150
FALLDGARGG PPEAFTTSVR SYLPNTVTDA LRGSGAWGLL LRRVGDDVLV
              160 170 180 190 200
HLLARCALFV LVAPSCAYQV CGPPLYQLGA ATQARPPPHA SGPRRRLGCE
              210 220 230 240 250
RAWNHSVREA GVPLGLPAPG ARRRGGSASR SLPLPKRPRR GAAPEPERTP
              260 270 280 290 300
VGQGSWAHPG RTRGPSDRGF CVVSPARPAE EATSLEGALS GTRHSHPSVG
              310 320 330 340 350
RQHHAGPPST SRPPRPWDTP CPPVYAETKH FLYSSGDKEQ LRPSFLLSSL
              360 370 380 390 400
RPSLTGARRL VETIFLGSRP WMPGTPRRLP RLPQRYWQMR PLFLELLGNH
              410 420 430 440 450
AQCPYGVLLK THCPLRAAVT PAAGVCAREK PQGSVAAPEE EDTDPRRLVQ
              460 470 480 490 500
LLRQHSSPWQ VYGFVRACLR RLVPPGLWGS RHNERRFLRN TKKFISLGKH
              510 520 530 540 550
AKLSLQELTW KMSVRDCAWL RRSPGVGCVP AAEHRLREEI LAKFLHWLMS
              560 570 580 590 600
VYVVELLRSF FYVTETTFQK NRLFFYRKSV WSKLQSIGIR QHLKRVQLRE
              610 620 630 640 650
LSEAEVRQHR EARPALLTSR LRFIPKPDGL RPIVNMDYVV GARTERREKR
              660 670 680 690 700
AERLTSRVKA LESVLNYERA RRPGLLGASV LGLDDIHRAW RTFVLRVRAQ
              710 720 730 740 750
DPPPELYFVK VDVTGAYDTI PQDRLTEVIA SIIKPQNTYC VRRYAVVQKA
              760 770 780 790 800
AHGHVRKAFK SHVLRPVPGD PAGLHPLHAA LQPVLRRHGE QAVCGDSAGR
AAPAFGG
hTERT isoform 3
SEQ ID NO. 3
                10 20 30 40 50
MPRAPRCRAV RSLLRSHYRE VLPLATFVRR LGPQGWRLVQ RGDPAAFRAL
                60 70 80 90 100
VAQCLVCVPW DARPPPAAPS FRQVSCLKEL VARVLQRLCE RGAKNVLAFG
              110 120 130 140 150
FALLDGARGG PPEAFTTSVR SYLPNTVTDA LRGSGAWGLL LRRVGDDVLV
              160 170 180 190 200
HLLARCALFV LVAPSCAYQV CGPPLYQLGA ATQARPPPHA SGPRRRLGCE
              210 220 230 240 250
RAWNHSVREA GVPLGLPAPG ARRRGGSASR SLPLPKRPRR GAAPEPERTP
              260 270 280 290 300
VGQGSWAHPG RTRGPSDRGF CVVSPARPAE EATSLEGALS GTRHSHPSVG
              310 320 330 340 350
RQHHAGPPST SRPPRPWDTP CPPVYAETKH FLYSSGDKEQ LRPSFLLSSL
              360 370 380 390 400
RPSLTGARRL VETIFLGSRP WMPGTPRRLP RLPQRYWQMR PLFLELLGNH
              410 420 430 440 450
AQCPYGVLLK THCPLRAAVT PAAGVCAREK PQGSVAAPEE EDTDPRRLVQ
              460 470 480 490 500
LLRQHSSPWQ VYGFVRACLR RLVPPGLWGS RHNERRFLRN TKKFISLGKH
              510 520 530 540 550
AKLSLQELTW KMSVRDCAWL RRSPGVGCVP AAEHRLREEI LAKFLHWLMS
              560 570 580 590 600
VYVVELLRSF FYVTETTFQK NRLFFYRKSV WSKLQSIGIR QHLKRVQLRE
              610 620 630 640 650
LSEAEVRQHR EARPALLTSR LRFIPKPDGL RPIVNMDYVV GARTERREKR
              660 670 680 690 700
AERLTSRVKA LESVLNYERA RRPGLLGASV LGLDDIHRAW RTFVLRVRAQ
              710 720 730 740 750
DPPPELYFVK VDVTGAYDTI PQDRLTEVIA SIIKPQNTYC VRRYAVVQKA
              760 770 780 790 800
AHGHVRKAFK SHVSTLTDLQ PYMRQFVAHL QETSPLRDAV VIEQSSSLNE
              810 820 830 840 850
ASSGLFDVFL REMCHHAVRI RGKSYVQCQG IPQGSILSTL LCSLCYGDME
              860 870 880 890 900
NKLFAGIRRD GLLLRLVDDF LLVTPHLTHA KTFLSYARTS IRASLTENRG
              910 920 930 940 950
FKAGRNMRRK LFGVLRLKCH SLFLDLQVNS LQTVCTNIYK ILLLQAYRFH
              960 970 980 990 1000
ACVLQLPFHQ QVWKNPTFFL RVISDTASLC YSILKAKNAG MSLGAKGAAG
          1010 1020 1030 1040 1050
PLPSEAVQWL CHQAFLLKLT RHRVTYVPLL GSLRTAQTQL SRKLPGTTLT
            1060
ALEAAANPAL PSDFKTILD
hTERT isoform 4
SEQ ID NO. 4
                10 20 30 40 50
MPRAPRCRAV RSLLRSHYRE VLPLATFVRR LGPQGWRLVQ RGDPAAFRAL
                60 70 80 90 100
VAQCLVCVPW DARPPPAAPS FRQVSCLKEL VARVLQRLCE RGAKNVLAFG
              110 120 130 140 150
FALLDGARGG PPEAFTTSVR SYLPNTVTDA LRGSGAWGLL LRRVGDDVLV
              160 170 180 190 200
HLLARCALFV LVAPSCAYQV CGPPLYQLGA ATQARPPPHA SGPRRRLGCE
              210 220 230 240 250
RAWNHSVREA GVPLGLPAPG ARRRGGSASR SLPLPKRPRR GAAPEPERTP
              260 270 280 290 300
VGQGSWAHPG RTRGPSDRGF CVVSPARPAE EATSLEGALS GTRHSHPSVG
              310 320 330 340 350
RQHHAGPPST SRPPRPWDTP CPPVYAETKH FLYSSGDKEQ LRPSFLLSSL
              360 370 380 390 400
RPSLTGARRL VETIFLGSRP WMPGTPRRLP RLPQRYWQMR PLFLELLGNH
              410 420 430 440 450
AQCPYGVLLK THCPLRAAVT PAAGVCAREK PQGSVAAPEE EDTDPRRLVQ
              460 470 480 490 500
LLRQHSSPWQ VYGFVRACLR RLVPPGLWGS RHNERRELRN TKKFISLGKH
              510 520 530 540 550
AKLSLQELTW KMSVRDCAWL RRSPGVGCVP AAEHRLREEI LAKFLHWLMS
              560 570 580 590 600
VYVVELLRSF FYVTETTFQK NRLFFYRKSV WSKLQSIGIR QHLKRVQLRE
              610 620 630 640 650
LSEAEVRQHR EARPALLTSR LRFIPKPDGL RPIVNMDYVV GARTERREKR
              660 670 680 690 700
AERLTSRVKA LFSVLNYERA RRPGLLGASV LGLDDIHRAW RTFVLRVRAQ
              710 720 730 740 750
DPPPELYFVK DRLTEVIASI IKPQNTYCVR RYAVVQKAAH GHVRKAFKSH
              760 770 780 790
VLRPVPGDPA GLHPLHAALQ PVLRRHGEQA VCGDSAGRAA PAFGG
```

### EXAMPLES

Certain aspects and embodiments of the invention will now be illustrated by way of example and with reference to the description, figures and tables set out herein. Such examples of the methods, uses and other aspects of the present invention are representative only, and should not be taken to limit the scope of the present invention to only such representative examples.

The examples show:

### Example 1: Generation of Immortalised MSCs

Mesenchymal Stromal Cells (MSCs) whose MSC-EVs repeatedly showed immunomodulatory activities in functional in vitro procedures were selected for immortalization. To identify the MSCs to be immortalized MSCs, the inventors used the modified mixed lymphocyte-Reaction (MLR) assay in accordance with the selection method disclosed in WO 2014/013029. Hence, only highly immune modulatory vesicle producing MSCs were used for immortalization.

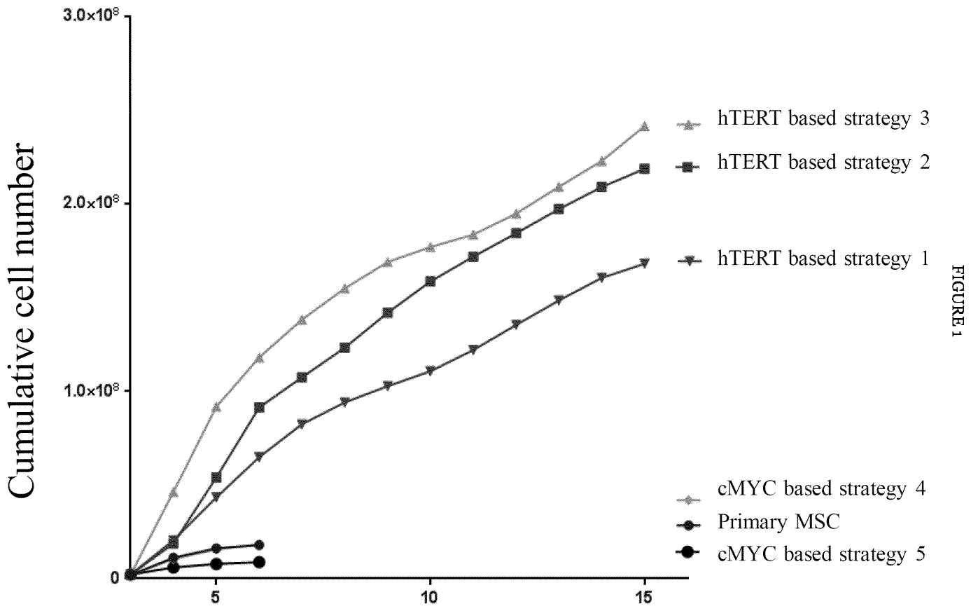

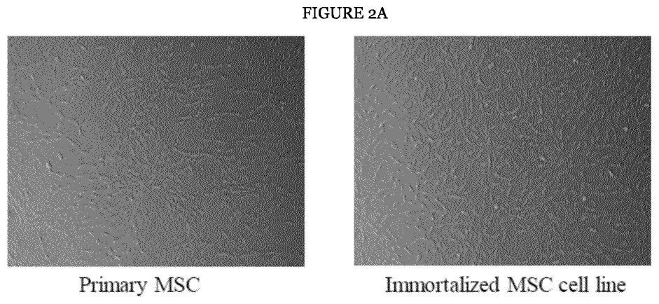

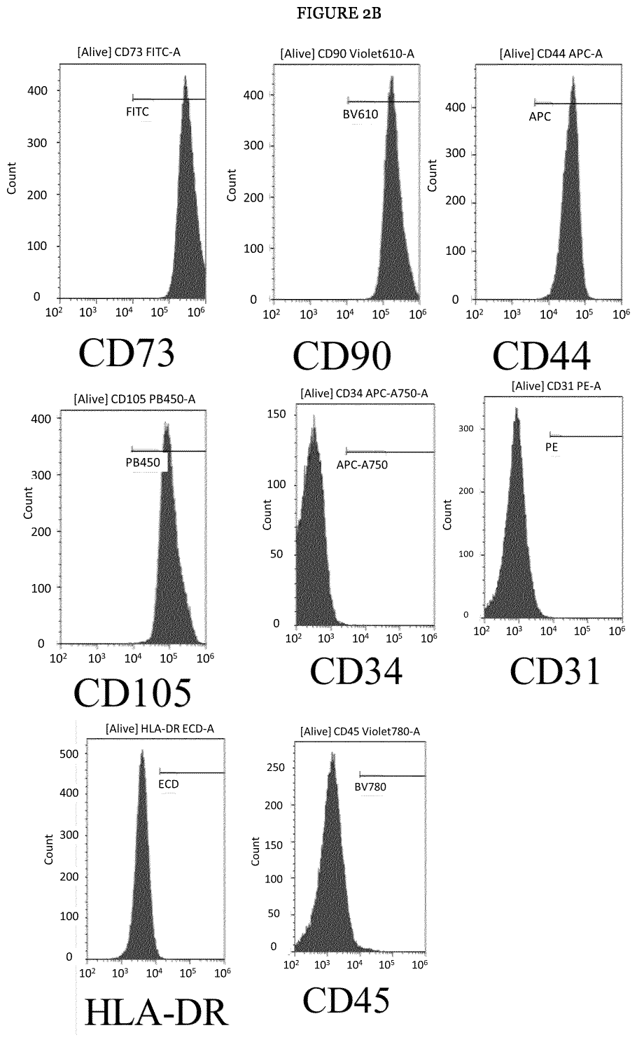

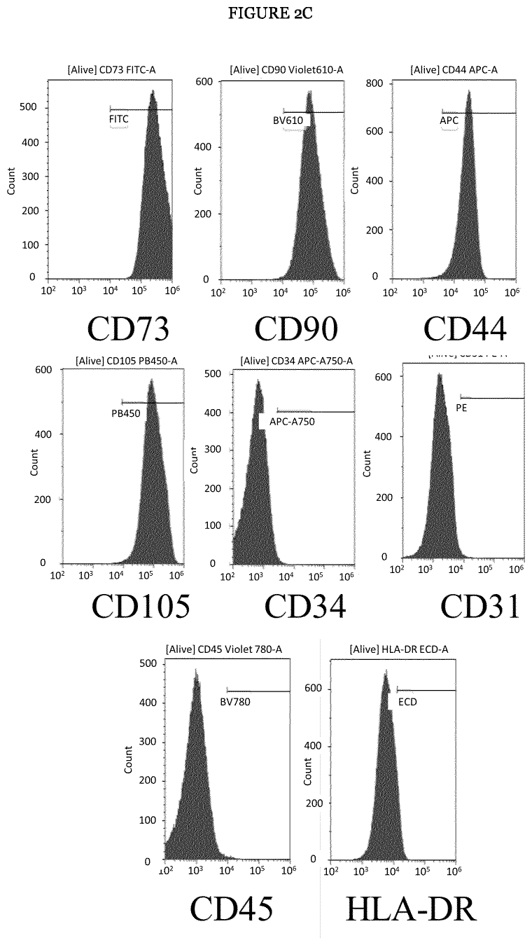

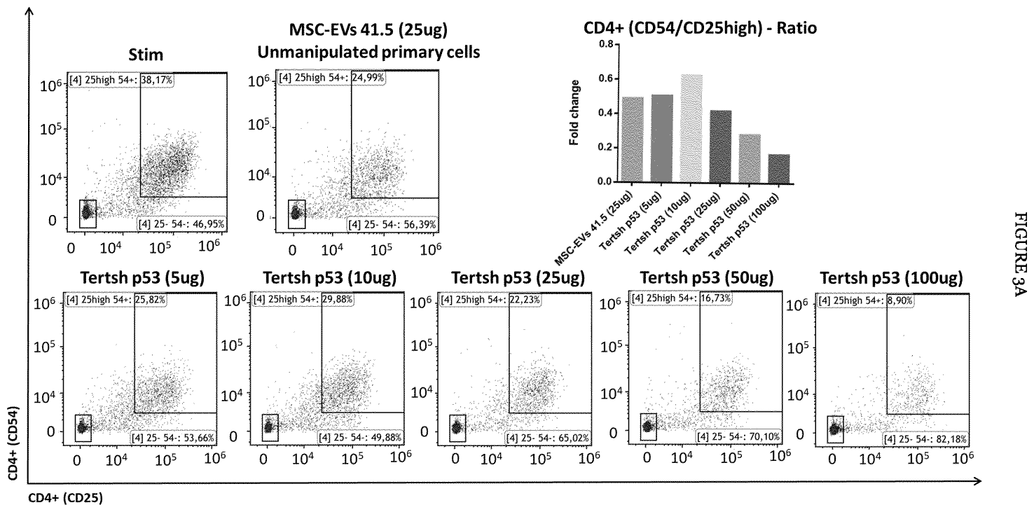

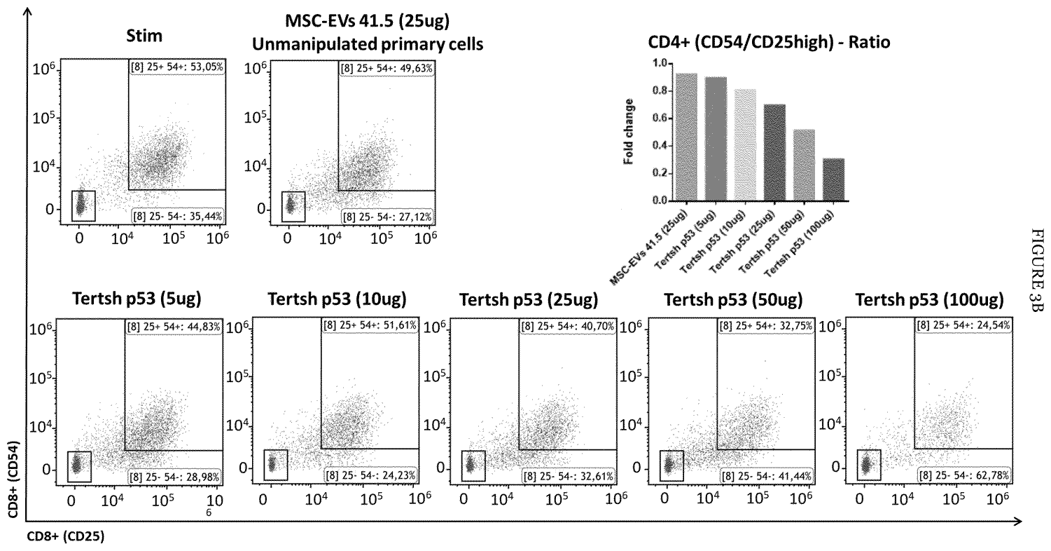

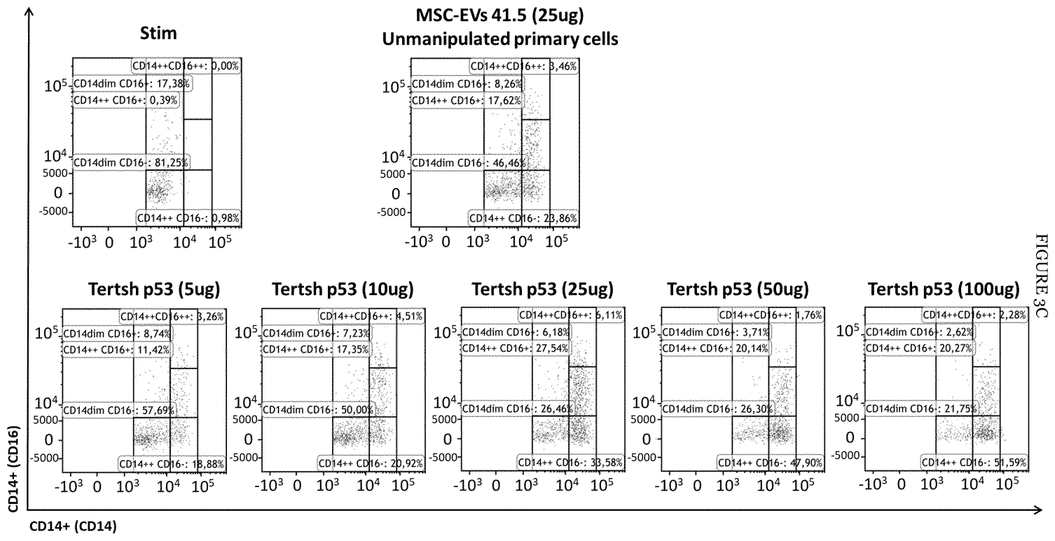

Several expression constructs were used to induce immortalization, in particular hTERT, and shRNA constructs for the targeted inhibition of p16, p21, or p53. The immortalization by lentiviral constructs coding only for hTERT (strategy 1) is inferior to strategies in which, in addition to the overexpression of TERT, an shRNA against p53 (strategy 2) or p21 (strategy 3) is introduced into the cells by means of the lentiviral transgene cassette, both in terms of the frequency of the immortalized cells obtained and the expansion rate of the resulting immortalized MSC lines shown in FIG. 1. As shown in FIG. 2A neither the morphology of the cells nor their cell surface phenotypes (FIGS. 2B and C) nor their differentiation potential is noticeably altered. In contrast, it was found that the morphology of cells changed significantly after attempted immortalization using transgenic c-myc expression. The findings clearly indicate that shRNA-encoding expression cassettes introduced in parallel with TERT significantly promote the immortalization process of MSCs and probably other immortalizable cells.

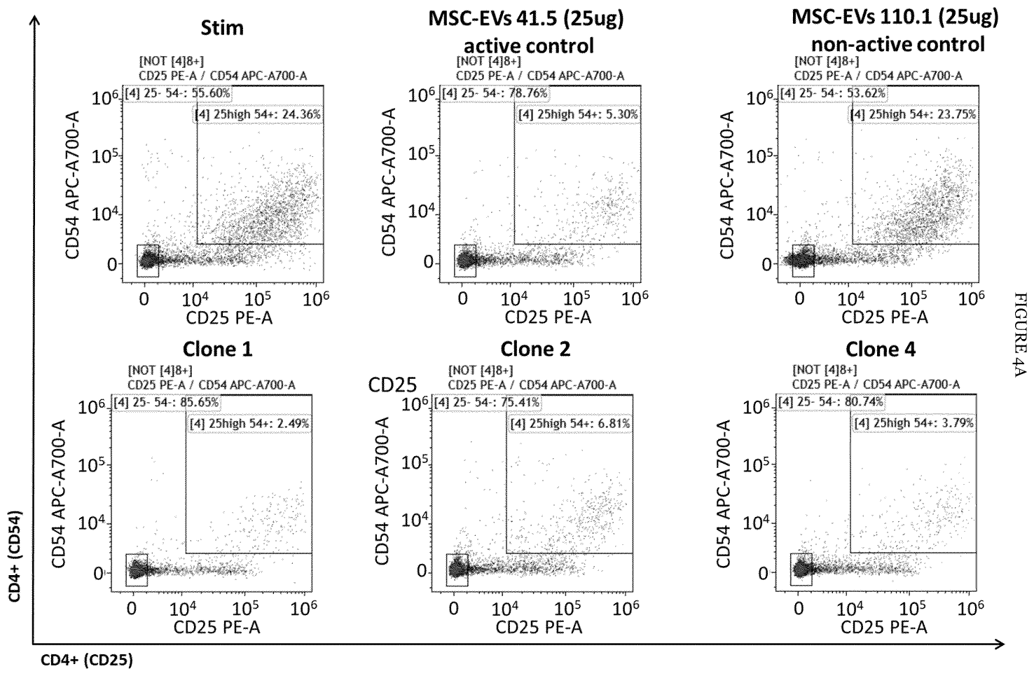

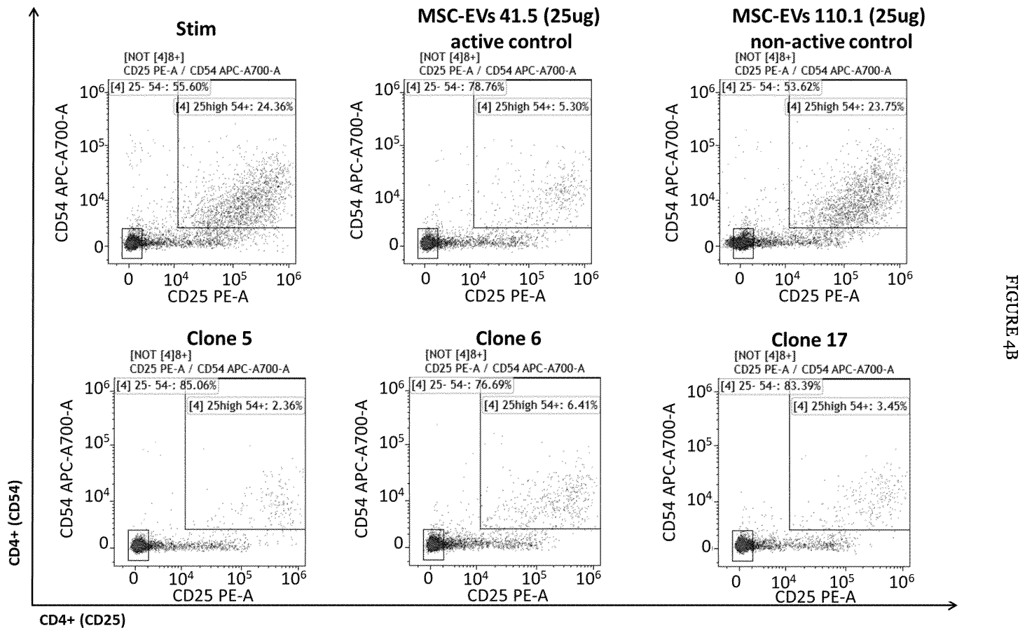

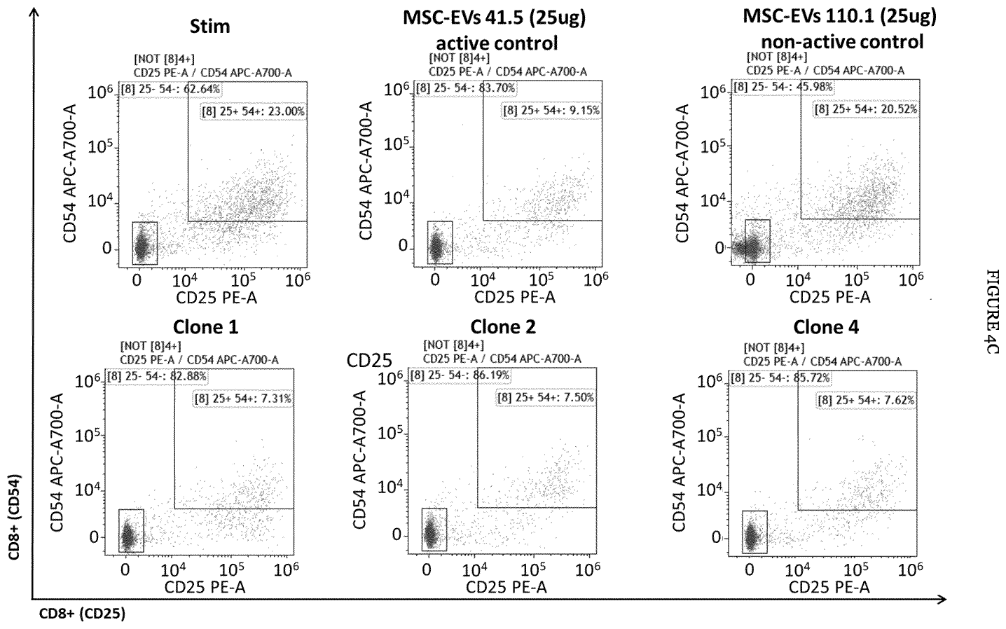

After immortalization, the EVS of the resulting cell lines were prepared and tested in the MLR assay as described above. According to the primary cells, not all immortalized MSCs secrete immunomodulatory active EVS and the activity was mostly weaker than in the EV preparations of corresponding primary MSCs (FIG. 3).

### Example 2: Generation of Clonal Immortalised MSCs

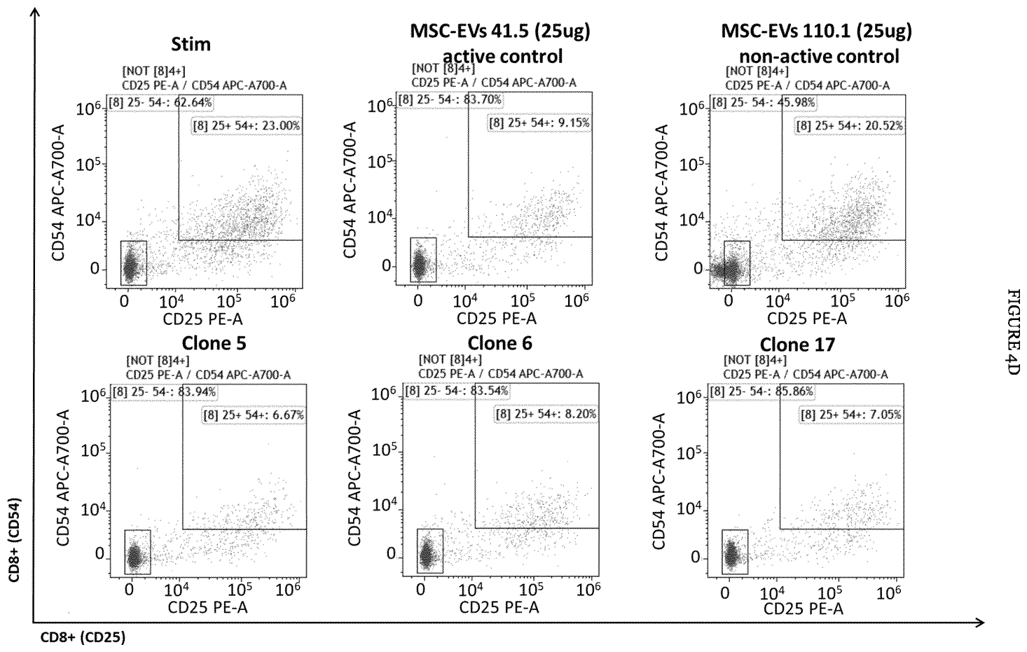

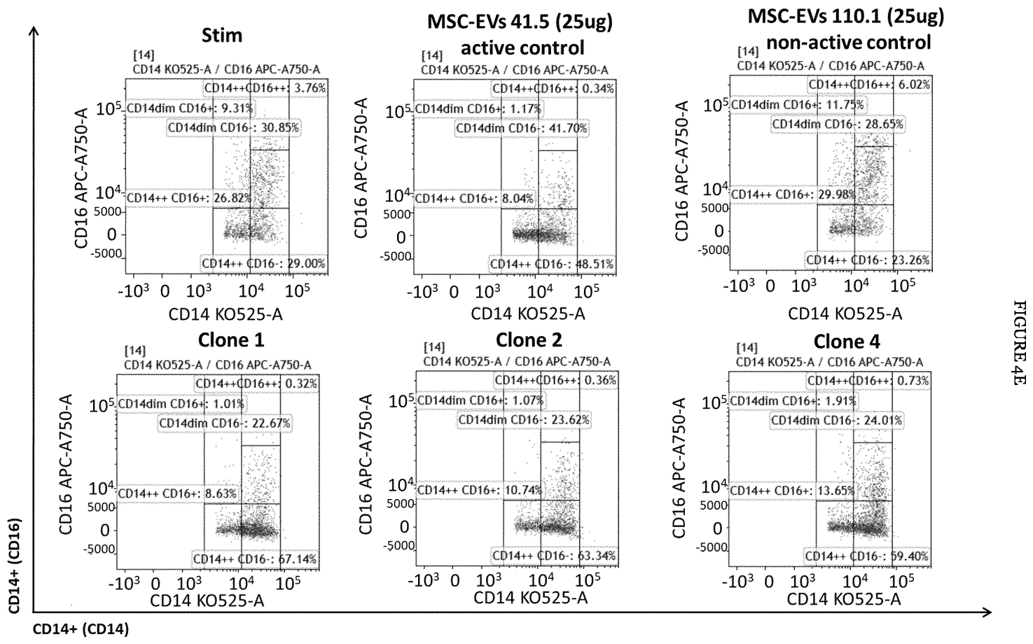

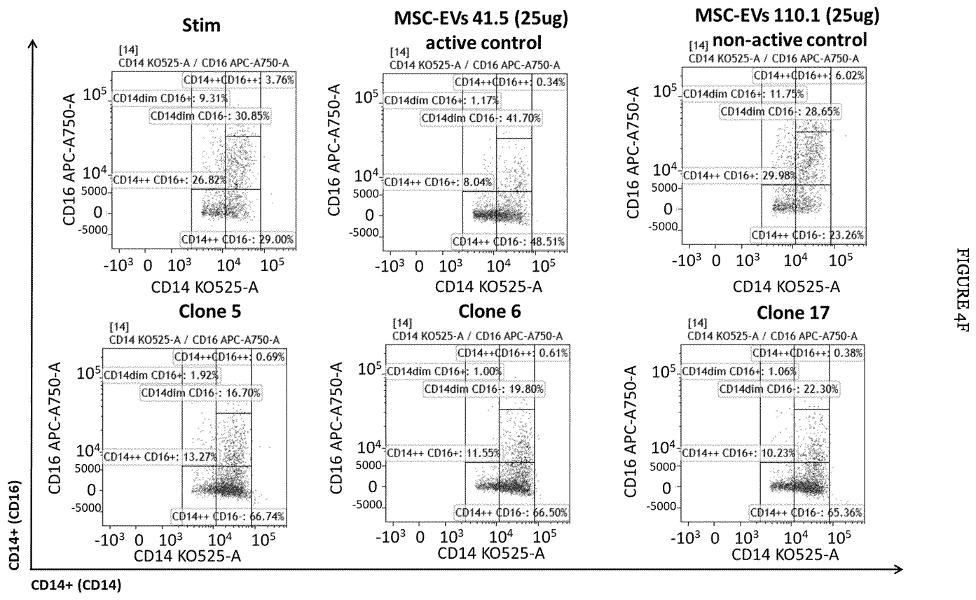

Another strategy was established in which the inventors deposited cells of immortalized MSC lines as single cells by flow cytometric cell sorting and produced clonal cell lines therefrom. Clonal selection of single cells is only possible if the cells are propagated in conditioned MSC medium. Indeed, this method allowed the production of different clonal MSC lines, whose EVs were prepared and then functionally tested in vitro. In this assay, it was observed that some of the EV preparations of the clonal MSCS have very high immunomodulatory activity, far exceeding the activity of the parent polyclonal line (FIG. 4).

### Example 3: Testing the Immunomodulatory Potential of MSC-EV Preparations

A mixed lymphocyte reaction (MLR) assay was performed. For this, MLR cells were thawed in (RPMI+10% hAB serum+1% PS+1:10000 benzonase) dropwise while shaking the 15 ml falcon tube containing the cells. The sample was centrifuge at 900 g for 5 min. The resulting pellet was resuspended in 1 ml of cultivation medium RPMI+10% hAB serum+1% PS. Subsequently, the obtained cells were counted and diluted to yield 600.000 cells/100 ul. Of this stock, 100 ul was put in separate wells. To the respective wells, 25 ug of MSC-EVs were added and 10 the volume filled up to 200 ul with cultivation media. This mixture was incubated for 5 days before data was evaluated.

1. An in vitro method for immortalising a target cell with a finite life-span, comprising the following steps:
(i) providing the target cell with a finite life-span,
(ii) contacting the target cell with a finite life-span with at least:
(a) a human Telomerase reverse transcriptase (hTERT) protein, or a genetic expression construct encoding a hTERT protein, and
(b) one or more inhibitors of the expression, function and/or stability of a cell cycle regulator/tumour suppressor protein chosen from a list comprising p21, p16 and/or p53;
Wherein the target cell with a finite life-span is contacted with at least (a) and (b) for a time sufficient to transform the target cell with a finite life-span into an immortalised target cell.

2. The method of claim 1, further comprising separating one or more immortalised target cell from the target cell with a finite life-span comprised in a mixture of target cell by passaging the mixture of target cell for at least 10 times.

3. The method of claim 1, wherein the target cell with finite life-span in step (i) is a mammalian cell, preferably a human cell, that is obtained from biological material including but not limited to bone marrow, adipose tissue, umbilical cord blood, umbilical cord tissue, placenta tissue, periodontal ligament, trabecular bone, synovial membrane, periosteum, muscle and skin tissue.

4. The method of claim 1, wherein in step (ii) the inhibitor of expression is an antisense construct, preferably an antisense construct comprising a sequence hybridizing to an mRNA of the at least one cell cycle regulator/tumour suppressor protein.

5. The method of claim 4, wherein the antisense construct is selected from an RNA interference inducing nucleic acid, such as an siRNA or shRNA, and preferably is an shRNA.

6. The method of claim 5, wherein the shRNA is provided by introducing into the target cell with finite life-span an shRNA expression construct.

7. The method of claim 6, wherein the genetic expression constructs encoding the hTERT protein and the at least one inhibitor of the expression, function and/or stability of a cell cycle regulator/tumour suppressor protein are inducible genetic expression constructs.

8. The method of claim 7, wherein the at least two inducible genetic expression constructs are controlled by a tetracycline-controlled operator system.

9. The method of claim 1, wherein one or more expression construct is stably or transiently introduced into the target cell with finite life-span.

10. The method of claim 1, further comprising after step (ii) an optional step (iii) of terminating or slowing down proliferation, inducing senescence and/or inducing differentiation in the immortalised target cell.

11. An immortalised target cell or immortalised target cell line obtainable with the method of claim 1.

12. A method for producing a therapeutically active target cell-derived extracellular vesicle (EV) preparation, the method comprising:
(i) Providing an immortalised target cell or immortalised target cell line by immortalising a target cell according to the method of claim 1;
(ii) Culturing the immortalised target cell or immortalised target cell line with a culture medium that allow for a secretion of therapeutically modular target cell EVs into the culture medium to obtain an EV enriched medium;
(iii) Harvesting from the EV enriched culture medium the EVs to obtain the therapeutic target cell-derived EV preparation;
(iv) Optionally, testing the therapeutic activity of the therapeutic target cell-derived EV preparation.

13. A vector or vector set for immortalisation of target cells with a finite life span, the vector or vector set comprising at least the following genetic elements (i) an expression cassette comprising expression control sequences operably linked to at least one hTERT gene, and (ii) an expression cassette comprising expression control sequences operably linked to at least one shRNA-coding sequence, wherein the shRNA targets an mRNA selected from (x) a p21 mRNA, (y) a p16 mRNA, and (z) a p53 mRNA.

14. A method of manufacturing a medicament that is immunomodulatory/immunoregulatory, anti-inflammatory, anti-fibrotic, angiogenetic, and/or wherein the medicament is for a regenerative treatment of an injury or a disease, comprising the steps of the method for immortalising a target cell with a finite life-span of claim 1.

15. The method of claim 14, wherein the medicament comprises one or more EVs obtained from the immortalised target cell or immortalised target cell line

16. The method of claim 15, wherein the EVs are obtained, by performing the a method comprising:
(i) Providing an immortalised target cell or immortalised target cell line by immortalising a target cell according to the method of claim 1;
(ii) Culturing the immortalised target cell or immortalised target cell line with a culture medium that allow for a secretion of therapeutically modular target cell EVs into the culture medium to obtain an EV enriched medium;
(iii) Harvesting from the EV enriched culture medium the EVs to obtain the therapeutic target cell-derived EV preparation;
(iv) Optionally, testing the therapeutic activity of the therapeutic target cell-derived EV preparation.


SEQUENCE LISTING (4 sequences)

SEQ ID NO: 1 - protein, 1132 aa, Homo sapiens
MPRAPRCRAVRSLLRSHYREVLPLATFVRRLGPQGWRLVQRGDPAAFRALVAQCLVCVPWDARPPPAAPSFRQVSCLKELVARVLQRLCERGAKNVLAFGFALLDGARGGPPEAFTTSVRSYLPNTVTDALRGSGAWGLLLRRVGDDVLVHLLARCALFVLVAPSCAYQVCGPPLYQLGAATQARPPPHASGPRRRLGCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLSSLRPSLTGARRLVETIFLGSRPWMPGTPRRLPRLPQRYWQMRPLFLELLGNHAQCPYGVLLKTHCPLRAAVTPAAGVCAREKPQGSVAAPEEEDTDPRRLVQLLRQHSSPWQVYGFVRACLRRLVPPGLWGSRHNERRFLRNTKKFISLGKHAKLSLQELTWKMSVRDCAWLRRSPGVGCVPAAEHRLREEILAKFLHWLMSVYVVELLRSFFYVTETTFQKNRLFFYRKSVWSKLQSIGIRQHLKRVQLRELSEAEVRQHREARPALLTSRLRFIPKPDGLRPIVNMDYVVGARTFRREKRAERLTSRVKALFSVLNYERARRPGLLGASVLGLDDIHRAWRTFVLRVRAQDPPPELYFVKVDVTGAYDTIPQDRLTEVIASIIKPQNTYCVRRYAVVQKAAHGHVRKAFKSHVSTLTDLQPYMRQFVAHLQETSPLRDAVVIEQSSSLNEASSGLFDVFLRFMCHHAVRIRGKSYVQCQGIPQGSILSTLLCSLCYGDMENKLFAGIRRDGLLLRLVDDFLLVTPHLTHAKTFLRTLVRGVPEYGCVVNLRKTVVNFPVEDEALGGTAFVQMPAHGLFPWCGLLLDTRTLEVQSDYSSYARTSIRASLTFNRGFKAGRNMRRKLFGVLRLKCHSLFLDLQVNSLQTVCTNIYKILLLQAYRFHACVLQLPFHQQVWKNPTFFLRVISDTASLCYSILKAKNAGMSLGAKGAAGPLPSEAVQWLCHQAFLLKLTRHRVTYVPLLGSLRTAQTQLSRKLPGTTLTALEAAANPALPSDFKTILD

SEQ ID NO: 2 - protein, 807 aa, Homo sapiens
MPRAPRCRAVRSLLRSHYREVLPLATFVRRLGPQGWRLVQRGDPAAFRALVAQCLVCVPWDARPPPAAPSFRQVSCLKELVARVLQRLCERGAKNVLAFGFALLDGARGGPPEAFTTSVRSYLPNTVTDALRGSGAWGLLLRRVGDDVLVHLLARCALFVLVAPSCAYQVCGPPLYQLGAATQARPPPHASGPRRRLGCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLSSLRPSLTGARRLVETIFLGSRPWMPGTPRRLPRLPQRYWQMRPLFLELLGNHAQCPYGVLLKTHCPLRAAVTPAAGVCAREKPQGSVAAPEEEDTDPRRLVQLLRQHSSPWQVYGFVRACLRRLVPPGLWGSRHNERRFLRNTKKFISLGKHAKLSLQELTWKMSVRDCAWLRRSPGVGCVPAAEHRLREEILAKFLHWLMSVYVVELLRSFFYVTETTFQKNRLFFYRKSVWSKLQSIGIRQHLKRVQLRELSEAEVRQHREARPALLTSRLRFIPKPDGLRPIVNMDYVVGARTFRREKRAERLTSRVKALFSVLNYERARRPGLLGASVLGLDDIHRAWRTFVLRVRAQDPPPELYFVKVDVTGAYDTIPQDRLTEVIASIIKPQNTYCVRRYAVVQKAAHGHVRKAFKSHVLRPVPGDPAGLHPLHAALQPVLRRHGEQAVCGDSAGRAAPAFGG

SEQ ID NO: 3 - protein, 1069 aa, Homo sapiens
MPRAPRCRAVRSLLRSHYREVLPLATFVRRLGPQGWRLVQRGDPAAFRALVAQCLVCVPWDARPPPAAPSFRQVSCLKELVARVLQRLCERGAKNVLAFGFALLDGARGGPPEAFTTSVRSYLPNTVTDALRGSGAWGLLLRRVGDDVLVHLLARCALFVLVAPSCAYQVCGPPLYQLGAATQARPPPHASGPRRRLGCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLSSLRPSLTGARRLVETIFLGSRPWMPGTPRRLPRLPQRYWQMRPLFLELLGNHAQCPYGVLLKTHCPLRAAVTPAAGVCAREKPQGSVAAPEEEDTDPRRLVQLLRQHSSPWQVYGFVRACLRRLVPPGLWGSRHNERRFLRNTKKFISLGKHAKLSLQELTWKMSVRDCAWLRRSPGVGCVPAAEHRLREEILAKFLHWLMSVYVVELLRSFFYVTETTFQKNRLFFYRKSVWSKLQSIGIRQHLKRVQLRELSEAEVRQHREARPALLTSRLRFIPKPDGLRPIVNMDYVVGARTFRREKRAERLTSRVKALFSVLNYERARRPGLLGASVLGLDDIHRAWRTFVLRVRAQDPPPELYFVKVDVTGAYDTIPQDRLTEVIASIIKPQNTYCVRRYAVVQKAAHGHVRKAFKSHVSTLTDLQPYMRQFVAHLQETSPLRDAVVIEQSSSLNEASSGLFDVFLRFMCHHAVRIRGKSYVQCQGIPQGSILSTLLCSLCYGDMENKLFAGIRRDGLLLRLVDDFLLVTPHLTHAKTFLSYARTSIRASLTFNRGFKAGRNMRRKLFGVLRLKCHSLFLDLQVNSLQTVCTNIYKILLLQAYRFHACVLQLPFHQQVWKNPTFFLRVISDTASLCYSILKAKNAGMSLGAKGAAGPLPSEAVQWLCHQAFLLKLTRHRVTYVPLLGSLRTAQTQLSRKLPGTTLTALEAAANPALPSDFKTILD

SEQ ID NO: 4 - protein, 795 aa, Homo sapiens
MPRAPRCRAVRSLLRSHYREVLPLATFVRRLGPQGWRLVQRGDPAAFRALVAQCLVCVPWDARPPPAAPSFRQVSCLKELVARVLQRLCERGAKNVLAFGFALLDGARGGPPEAFTTSVRSYLPNTVTDALRGSGAWGLLLRRVGDDVLVHLLARCALFVLVAPSCAYQVCGPPLYQLGAATQARPPPHASGPRRRLGCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLSSLRPSLTGARRLVETIFLGSRPWMPGTPRRLPRLPQRYWQMRPLFLELLGNHAQCPYGVLLKTHCPLRAAVTPAAGVCAREKPQGSVAAPEEEDTDPRRLVQLLRQHSSPWQVYGFVRACLRRLVPPGLWGSRHNERRFLRNTKKFISLGKHAKLSLQELTWKMSVRDCAWLRRSPGVGCVPAAEHRLREEILAKFLHWLMSVYVVELLRSFFYVTETTFQKNRLFFYRKSVWSKLQSIGIRQHLKRVQLRELSEAEVRQHREARPALLTSRLRFIPKPDGLRPIVNMDYVVGARTFRREKRAERLTSRVKALFSVLNYERARRPGLLGASVLGLDDIHRAWRTFVLRVRAQDPPPELYFVKDRLTEVIASIIKPQNTYCVRRYAVVQKAAHGHVRKAFKSHVLRPVPGDPAGLHPLHAALQPVLRRHGEQAVCGDSAGRAAPAFGG